SEQUENCE LISTING (25 sequences)

SEQ ID NO: 1 - protein, 117 aa, synthetic construct
EVQLQQSGPELVKPGSSVKISCKGSGYTFTDYNMEWVKQSHGKSLEWIGVINPNNRFTSYNQNFRGKATLTVDKSSSTAYMDLRSLTSEDSAVYFCAGSRWYQYDYWGQGTTLTVSS

SEQ ID NO: 2 - protein, 117 aa, synthetic construct
QVQLVQSGAEVKKPGASVKVSCKASGYTFTDYNMEWVRQAPGQGLEWMGVINPNNRFTSYNQNFRGRVTMTRDTSTSTVYMELSSLRSEDTAVYYCARSRWYQYDYWGQGTLVTVSS

SEQ ID NO: 3 - protein, 117 aa, synthetic construct
QVQLVQSGAEVKKPGASVKVSCKASGYTFTDYNMEWVRQAPGQRLEWMGVINPNNRFTSYNQNFRGRVTITRDTSASTAYMELSSLRSEDTAVYYCARSRWYQYDYWGQGTLVTVSS

SEQ ID NO: 4 - protein, 117 aa, synthetic construct
QVQLVQSGAEVKKPGSSVKVSCKASGYTFTDYNMEWVRQAPGQGLEWMGVINPNNRFTSYNQNFRGRVTITADKSTSTAYMELSSLRSEDTAVYYCARSRWYQYDYWGQGTLVTVSS

SEQ ID NO: 5 - protein, 117 aa, synthetic construct
QVQLVQSGSELKKPGASVKVSCKASGYTFTDYNMEWVRQAPGQGLEWMGVINPNNRFTSYNQNFRGRFVFSLDTSVSTAYLQISSLKAEDTAVYYCARSRWYQYDYWGKGTTVTVSS

SEQ ID NO: 6 - protein, 117 aa, synthetic construct
EVQLVQSGAEVKKPGESLKISCKGSGYTFTDYNMEWVRQMPGKGLEWMGVINPNNRFTSYNQNFRGQVTISADKSISTAYLQWSSLKASDTAMYYCARSRWYQYDYWGQGTLVTVSS

SEQ ID NO: 7 - protein, 109 aa, synthetic construct
QVVVTQESALTTSPGETVTLTCRSSTGAVTTSNYANWVQEKPDHLFTGLIGGINNRAPGVPARFSGSLIGDKAALTITGAQTEDEAIYFCALWYSNHWVFGGGTKLTVL

SEQ ID NO: 8 - protein, 99 aa, synthetic construct
QAVVTQEPSLTVRSSTGAVTTSNYANWFQQKPGQAPRTLIYGINNRAPWTPARFSGSLLGGKAALTLSGAQPEDEAEYYCALWYSNHWVFGGGTKLTVL

SEQ ID NO: 9 - protein, 109 aa, synthetic construct
QTVVTQEPSLTVSPGGTVTLTCRSSTGAVTTSNYANWFQQKPGQAPRALIYGINNRAPWTPARFSGSLLGGKAALTLSGVQPEDEAEYYCALWYSNHWVFGTGTKVTVL

SEQ ID NO: 10 - protein, 109 aa, synthetic construct
QTVVTQEPSFSVSPGGTVTLTCRSSTGAVTTSNYANWYQQTPGQAPRTLIYGINNRAPGVPDRFSGSILGNKAALTITGAQADDESDYYCALWYSNHWVFGGGTKLTVL

SEQ ID NO: 11 - protein, 110 aa, synthetic construct
EIVMTQSPATLSVSPGERATLSCRSSTGAVTTSNYANWYQQKPGQAPRLLIYGINNRAPGIPARFSGSGSGTEFTLTISSLQSEDFAVYYCALWYSNHWVFGGGTKVEIK

SEQ ID NO: 12 - protein, 110 aa, synthetic construct
QSVLTQPPSASGTPGQRVTISCRSSTGAVTTSNYANWYQQLPGTAPKLLIYGINNRAPGVPDRFSGSKSGTSASLAISGLQSEDEADYYCALWYSNHWVFGGGTKLTVLG

SEQ ID NO: 13 - protein, 117 aa, synthetic construct
EVQLVQSGAEVKKPGESLKISCKGSGYTFTDYNMEWVRQMPGKGLEWMGVINPNNRFTSYNQNFRGQVTISADKSISTAYLQWSSLKASDTAMYYCAGSRWYQYDYWGQGTLVTVSS

SEQ ID NO: 14 - protein, 117 aa, synthetic construct
EVQLVQSGAEVKKPGESLKISCKGSGYTFTDYNMEWVRQMPGKGLEWIGVINPNNRFTSYNQNFRGQVTISADKSISTAYLQWSSLKASDTAMYFCAGSRWYQYDYWGQGTLVTVSS

SEQ ID NO: 15 - protein, 117 aa, synthetic construct
EVQLVQSGAEVKKPGESLKISCKGSGYTFTDYNMEWVRQMPGKGLEWIGVINPNNRFTSYNQNFRGQVTISADKSISTAYLQWSSLKASDTAMYYCAGSRWYQYDYWGQGTLVTVSS

SEQ ID NO: 16 - protein, 117 aa, synthetic construct
EVQLVQSGAEVKKPGESLKISCKGSGYTFTDYNMEWVRQMPGKGLEWMGVINPNNRFTSYNQNFRGQVTISADKSISTAYLQWSSLKASDTAMYFCAGSRWYQYDYWGQGTLVTVSS

SEQ ID NO: 17 - protein, 109 aa, synthetic construct
QTVVTQEPSLTVSPGGTVTLTCRSSTGAVTTSNYANWFQQKPGQAPRGLIGGINNRAPGTPARFSGSLLGGKAALTLSGVQPEDEAEYYCALWYSNHWVFGTGTKVTVL

SEQ ID NO: 18 - protein, 66 aa, synthetic construct
EVQLVQSGAEVKKPGESLKISCKGSGYTFTDYNMEWVRQMPGKGLEWMGVINPNNRFTSYNQNFRG

SEQ ID NO: 19 - protein, 66 aa, synthetic construct
EVQLQQSGPELVKPGSSVKISCKGSGYTFTDYNMEWVKQSHGKSLEWIGVINPNNRFTSYNQNFRG

SEQ ID NO: 20 - protein, 51 aa, synthetic construct
QVTISADKSISTAYLQWSSLKASDTAMYYCARSRWYQYDYWGQGTLVTVSS

SEQ ID NO: 21 - protein, 51 aa, synthetic construct
KATLTVDKSSSTAYMDLRSLTSEDSAVYFCAGSRWYQYDYWGQGTTLTVSS

SEQ ID NO: 22 - protein, 66 aa, synthetic construct
QTVVTQEPSLTVSPGGTVTLTCRSSTGAVTTSNYANWFQQKPGQAPRALIYGINNRAPWTPARFSG

SEQ ID NO: 23 - protein, 66 aa, synthetic construct
QVVVTQESALTTSPGETVTLTCRSSTGAVTTSNYANWVQEKPDHLFTGLIGGINNRAPGVPARFSG

SEQ ID NO: 24 - protein, 43 aa, synthetic construct
SLLGGKAALTLSGVQPEDEAEYYCALWYSNHWVFGTGTKVTVL

SEQ ID NO: 25 - protein, 43 aa, synthetic construct
SLIGDKAALTITGAQTEDEAIYFCALWYSNHWVFGGGTKLTVL

(19) United States
(12) Patent Application Publication
(10) Pub. No.: US 2024/0294615 A1
(43) Pub. Date: Sep. 5, 2024

(54) HUMANIZED ANTIBODIES AGAINST NEISSERIA GONORRHOEAE AND METHODS OF USE THEREOF

(71) Applicant: UNIVERSITY OF MASSACHUSETTS, Boston, MA (US)

(72) Inventors: Jutamas SHAUGHNESSY, Carlisle, MA (US); Peter A. RICE, Southborough, MA (US); Sunita GULATI, Spencer, MA (US); Sanjay RAM, Worcester, MA (US); Celia SCHIFFER, Boston, MA (US)

(21) Appl. No.: 18/408,793

(22) Filed: Jan. 10, 2024

Related U.S. Application Data
(60) Provisional application No. 63/448,137, filed on Feb. 24, 2023.
(60) Provisional application No. 63/438,518, filed on Jan. 11, 2023.

(51) Int. Cl. C07K 16/12; A61K 39/00; A61P 31/04

(52) U.S. Cl. CPC C07K 16/1217; A61P 31/04; A61K 2039/505; C07K 2317/24; C07K 2317/56; C07K 2317/734

(57) ABSTRACT

Provided are humanized antibodies that bind to the 2C7 epitope of Neisseria gonorrhoeae. Also provided are compositions and methods for treating Neisseria gonorrhoeae infection in a subject in need thereof.

### RELATED APPLICATIONS

The present invention claims the benefit of U.S. Provisional Application No. 63/438,518, filed Jan. 11, 2023 and U.S. Provisional Application No. 63/448,137, filed Feb. 24, 2023, the contents of which are incorporated herein by reference in their entireties for all purposes.

### STATEMENT OF FEDERALLY SPONSORED RESEARCH

This invention was made with government support under Grant Nos. AI136007 and AI132296 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML file, created on May 28, 2024, is named 748692_UM9-223CIPB_ST26.xml and is 24,927 bytes in size.

### FIELD

The disclosure relates to humanized variants of the mouse 2C7 monoclonal antibody directed against Neisseria gonorrhoeae and methods of use of the same.

### BACKGROUND

Neisseria gonorrhoeae is the second most common bacterial sexually transmitted infection (STI); the worldwide incidence is 82 million cases per year. Antimicrobials are used as conventional treatment for Neisseria gonorrhoeae infection. However, there has been a recent emergence of resistance to commonly used antimicrobials. With the potential for an era of untreatable Neisseria gonorrhoeae infections, there is an urgent need for novel treatments for Neisseria gonorrhoeae infection, specifically treatments that are not traditional antibiotics.

The 2C7 epitope of Neisseria gonorrhoeae is a sugar structure on the surface of gonococci that was previously identified as a promising target for preventing Neisseria gonorrhoeae infection, because gonococcal infection elicits a significant increase in IgG anti-2C7 epitope antibody in human subjects (Gulati, S. et al, J Infect Dis 1996 1223-1237). A mouse monoclonal antibody that binds the 2C7 epitope of Neisseria gonorrhoeae has been found to induce killing and phagocytosis of gonococci in in vitro studies. Previous studies have suggested that the increased bactericidal activity of the mouse 2C7 antibody is due to enhancing complement activation on bacteria (Gulati, S., PLoS Pathogens 2013 e1003559).

These in vitro studies, as well as the epitope being present on 94% of gonococci that reside in the human genital tract, make 2C7 a promising target for therapeutic antibodies (Gulati, S. et al., J Infect Dis 1996 1223-1237). However, the previously discovered 2C7 antibody is a murine antibody and, as such, is not suitable for repeat dosing in human subjects in view of the human anti-mouse antibody (HAMA) response elicited therein.

Thus, there is an urgent need in the art for non-antibiotic treatments of Neisseria gonorrhoeae. There is also a need in the art for humanized variants of murine antibodies that have potential therapeutic uses.

### SUMMARY

The present disclosure is based on the discovery of a humanized monoclonal antibody (mAb) that binds a specific epitope of Neisseria gonorrhoeae (e.g., 2C7) for treating Neisseria gonorrhoeae infection. In one aspect, the present disclosure provides a humanized antibody that binds the 2C7 epitope of Neisseria gonorrhoeae antigen, wherein the antibody comprises a variable heavy (VH) domain, a variable light (VL) domain, and a human Fc region, wherein the VH domain comprises an amino acid sequence selected from the group consisting of SEQ ID NOs: 13-16, and wherein the VL domain comprises an amino acid sequence set forth in SEQ ID NOs: 17.

In an embodiment of the humanized antibody, the VH domain comprises the amino acid sequence of SEQ ID NO: 13 and the VL domain comprises the amino acid sequence of SEQ ID NO: 17.

In an embodiment of the humanized antibody, the VH domain comprises the amino acid sequence of SEQ ID NO: 14 and the VL domain comprises the amino acid sequence of SEQ ID NO: 17.

In an embodiment of the humanized antibody, the VH domain comprises the amino acid sequence of SEQ ID NO: 15 and the VL domain comprises the amino acid sequence of SEQ ID NO: 17.

In an embodiment of the humanized antibody, the VH domain comprises the amino acid sequence of SEQ ID NO: 16 and the VL domain comprises the amino acid sequence of SEQ ID NO: 17.

In other embodiments of the humanized antibody, the human Fc region is an IgG3 Fc region.

In other embodiments of the humanized antibody, the human Fc region is an IgG3 Fc region comprising one or more mutation that alters one or more Fc function.

In other embodiments of the humanized antibody, the one or more Fc function is selected from the group consisting of Fc clustering, C1q binding, complement-dependent cytotoxicity (CDC), antibody-dependent cell-mediated cytotoxicity (ADCC), and antibody dependent cellular phagocytosis (ADCP).

In another embodiment of the humanized antibody, the one or more Fc mutation increases CDC.

In an aspect, provided herein are pharmaceutical compositions comprising the humanized antibodies disclosed herein and a pharmaceutically acceptable carrier or diluent.

In another aspect, provided herein is a nucleic acid encoding the amino acid sequence of the humanized antibodies disclosed herein.

In another aspect, provided herein is a vector comprising the nucleic acid disclosed herein.

In another aspect, provided herein is a host cell comprising the vector disclosed herein.

In an embodiment, the host cell is a prokaryotic cell or a eukaryotic cell.

In an embodiment, the prokaryotic cell is an E. coli cell.

In an embodiment, the eukaryotic cell is selected from the group consisting of a yeast cell, a plant cell, an insect cell, and a mammalian cell.

In an embodiment, the plant cell is a tobacco plant cell.

In an embodiment, the mammalian cell is a human embryonic kidney (HEK) cell or a Chinese hamster ovary (CHO) cell.

In an embodiment, the host cell is a CHO cell.

In yet another aspect, provided herein are methods of producing a humanized antibody that binds the 2C7 epitope of Neisseria gonorrhoeae antigen, the method comprising the steps of culturing the host cell disclosed herein in culture medium under conditions sufficient to produce the humanized antibody.

In yet another aspect, provided herein are methods of treating Neisseria gonorrhoeae infection in a subject in need thereof comprising administering to the subject an antibody disclosed herein or the pharmaceutical compositions disclosed herein.

In an embodiment of the methods, the administered antibody or pharmaceutical composition improves clearance of the Neisseria gonorrhoeae infection.

In an embodiment of the methods, the administered antibody or pharmaceutical composition protects the subject from a subsequent Neisseria gonorrhoeae infection.

In another embodiment of the methods, the administered antibody or pharmaceutical composition improves clearance of the Neisseria gonorrhoeae infection and protects the subject from a subsequent Neisseria gonorrhoeae infection.

In another embodiment of the methods, the Neisseria gonorrhoeae infection is resistant to antibiotics.

In yet another embodiment of the methods, the subject is a human.

The summary of the disclosure described above is non-limiting and other features and advantages of the disclosed apparatus and methods will be apparent from the following drawings, detailed description of the disclosure, and claims.

### BRIEF DESCRIPTION OF THE DRAWINGS

The foregoing and other features and advantages of the present invention will be more fully understood from the following detailed description of illustrative embodiments taken in conjunction with the accompanying drawings. The file of this patent contains at least one drawing/photograph executed in color. Copies of this patent with color drawing(s)/photograph(s) will be provided by the Office upon request and payment of the necessary fee.

FIG. 1A-FIG. 1C show the selective survival of FA1090wt and FA1090lgtG⁻ mixed in equal proportions and inoculated into non-immune mice. FIG. 1A displays a Kaplan-Meier plot showing the clearance of FA1090wt and FA1090lgtG⁻ in mice. FIG. 1B displays a plot showing the colonization of FA1090wt and FA1090lgtG⁻ at daily intervals. FIG. 1C displays a dot plot showing the bacterial burdens of FA1090wt and FA1090lgtG⁻ over time.

FIG. 2 shows the binding of 20 humanized monoclonal antibody 2C7 molecules (H1L1, H1L2, H1L3, H1L4, H1L5, H3L1, H3L2, H3L3, H3L4, H3L5, H4L1, H4L2, H4L3, H4L4, H4L5, H5L1, H5L2, H5L3, H5L4, and H5L5) to gonococcal lipooligosaccharide (LOS) in an ELISA assay. The concentrations of the monoclonal antibodies are indicated on the X-axis and binding measured as optical density (OD) at 410 nm, indicated on the Y-axis. H0L0 indicates the chimeric monoclonal antibody 2C7.

FIG. 3 shows the binding to N. gonorrhoeae strain 15253 of 6 selected humanized monoclonal antibody 2C7 molecules (H0L0, H1L1, H1L2, H3L1, H3L2, H5L2, and H5L1) by flow cytometry. The concentrations of the monoclonal antibodies are indicated on the X-axis and binding measured as median fluorescence intensity is indicated on the Y-axis. H0L0 indicates the chimeric monoclonal antibody 2C7.

FIGS. 4A-4B (SEQ ID NO: 18-21) show the effect of VH5 back mutations on binding to N. gonorrhoeae strain 15253 of humanized 2C7 (H5L2 variants). FIG. 4A highlights amino acids in the humanized VH5 sequence selected for back mutation to the murine counterpart (VH sequence): the three backmutations created were G→S, M→I, and R→G. FIG. 4B shows the binding to N. gonorrhoeae strain 15253 of each of the humanized 2C7 monoclonal antibodies with the mouse VL2 sequence (abbreviated as L2) and humanized VH5 sequences either unmodified (H5L2) or with each of the three back mutations (highlighted in FIG. 4A and labeled in FIG. 4B as G→S, M→I, and R→G). A variant that had all the VL2 back mutations (VL2 back mutations are shown ahead in FIG. 5A) and each of the 3 VH5 back mutations were also created (H5 “all”-VL “all”, shown in FIG. 4B). All monoclonal antibodies were used at [20 μg/mL]. Chimeric monoclonal antibody 2C7 (labeled Chimeric 2C7-Umass) was used as a positive control. An IgG isotype control, detectable by anti-human IgG-FITC, was used as a negative control (labeled Control). The X-axis shows fluorescence, and the Y-axis shows the counts. Numbers alongside each histogram represent the medium fluorescence intensity of the entire bacterial population.

FIGS. 5A-5B (SEQ ID NO: 22-25) show the effects of VL2 back mutations on binding of humanized 2C7 (H5L2 variants) to N. gonorrhoeae strain 15253. FIG. 5A highlights the amino acids in the humanized VL2 sequence selected for back mutation to the murine counterpart (VL sequence). The five back mutations created were G→E, A→G, Y→G, W→G, and T→G. FIG. 5B shows the binding of each of the humanized 2C7 monoclonal antibodies with the VH5 sequence (abbreviated H5) and each of the humanized VL2 sequence either unmodified (H5L2) or with each of the five VL2 back mutations to N. gonorrhoeae strain 15253. Each monoclonal antibody was used at a concentration of 20 μg/mL. Chimeric monoclonal antibody 2C7 was used as a positive control (labeled Chimeric 2C7-UMass) and an IgG isotype control, detectable by anti-human IgG-FITC, was used as a negative control (labeled Control). The X axis shows fluorescence, and the Y axis shows the counts. Numbers alongside each histogram represent the median fluorescence intensity of the entire bacterial population.

FIG. 6 shows binding to N. gonorrhoeae strain 15253 of humanized 2C7 H5L2 with back-mutated H5 (R→G) in combination with each of the five individual L2 mutations (T→G, W→G, Y→G, A→G, and G→E,) either singly or simultaneously (L2 “all”). All humanized variants possessed the complement-enhancing substitution in Fc. Each monoclonal antibody was used at a concentration of 50 μg/mL. Chimeric monoclonal antibody 2C7 was used as a positive control (labeled Chimeric 2C7 (WT-Fc)-UMass) and an IgG isotype control, detectable by anti-human IgG-FITC, was used as a negative control (labeled Control). The X axis shows fluorescence, and the Y axis shows the counts. Numbers alongside each histogram represent the median fluorescence intensity of the entire bacterial population.

FIG. 7 (SEQ ID NO: 13-25) shows back mutations made to enhance activity of humanized monoclonal antibody 2C7; one in VH5 (R→G), three in VL2 (A→G, Y→G, and W→G) (shown with encircled arrows). VH and VL sequences are original mouse variable sequences while VH5 and VL2 are humanized counterparts. CDR amino acids are highlighted using rectangular boxes. Two additional back mutations (M→I and Y→F) made in VH5 are shown as arrows in square boxes.

FIG. 8 shows bactericidal activity directed against N. gonorrhoeae strain FA1090 of humanized monoclonal 2C7 IgG3 that also embodies the indicated VH5 mutation (R→G) plus the 3 VL2 mutations shown in FIG. 7 (A→G, Y→G, and W→G), designated here as VL2*** (VL2 with the A→G, Y→G, and W→G mutations). The individual VL2 variants that comprise VL2*** are shown schematically in FIG. 7 (circled arrows). Bacteria were incubated with increasing concentrations of antibody (indicated on the X-axis) and 20% complement (IgG/IgM depleted normal human serum): survival at 30 minutes relative to 0 minutes is indicated as % survival on the Y axis. Dose-responsive killing of N. gonorrhoeae was observed.

FIGS. 9A-9B show bactericidal activity against two strains of N. gonorrhoeae (FA1090 and 15253) of humanized monoclonal antibody 2C7 that possessed human IgG3 Fc. Bacteria were incubated with increasing concentrations of antibody (indicated on the X axis) and 20% complement (IgG/IgM depleted normal human serum): survival at 30-minutes relative to 0 minutes is indicated as % survival on the Y axis. Dose-responsive killing of N. gonorrhoeae was observed. FIG. 9A shows the bactericidal activity of N. gonorrhoeae strain FA1090; FIG. 9B shows the bactericidal activity of N. gonorrhoeae strain 15253.

FIGS. 10A-10C show the efficacy of humanized monoclonal antibody 2C7 (containing the H5 R→G and VL2 Y→G, A→G and W→G back mutations, also possessing human IgG3 Fc, against N. gonorrhoeae strain FA1090 in the mouse vaginal colonization model of gonorrhea. Premarin®-treated dual transgenic (human FH and C4BP) mice were given 2.2×10⁷ CFU strain FA1090 intravaginally on Day 0. Mice (n=10/group) were given either 3.3 or 10 μg of humanized monoclonal antibody 2C7 or PBS (control; n=10) intravenously on Day 1. FIG. 10A are Kaplan Meier curves showing time to clearance of infection. FIG. 10B Shows the log₁₀ CFU of N. gonorrhoeae versus time (days; X-axis). FIG. 10C shows bacterial burdens consolidated overtime (Area Under the Curve (AUC) [log₁₀ CFU] analysis) for each group of animals. Statistical comparisons (animals administered antibody vs. PBS) used the Mann-Whitney (nonparametric) test.

### DETAILED DESCRIPTION OF THE DISCLOSURE

The present disclosure is directed to a humanized variant of a mouse monoclonal antibody, which has binding specificity to the 2C7 epitope of Neisseria gonorrhoeae. In certain embodiments, the humanized antibodies have one or more mutations in the IgG3 Fc region that alters one or more Fc function.

The present disclosure is also directed to methods of treating Neisseria gonorrhoeae infection with a humanized antibody disclosed herein. In certain embodiments, a humanized antibody of the present invention is used to treat antibiotic resistant Neisseria gonorrhoeae.

It is to be understood that the methods described in this disclosure are not limited to particular methods and experimental conditions disclosed herein; as such methods and conditions may vary. It is also to be understood that the terminology used herein is for the purpose of describing particular embodiments only, and is not intended to be limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by MR Green and J. Sambrook and Harlow et al., Antibodies: A Laboratory Manual, Chapter 14, Cold Spring Harbor Laboratory, Cold Spring Harbor (2013, 2nd edition).

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including”, as well as other forms, such as “includes” and “included”, is not limiting. As used herein, unless otherwise stated, the singular forms “a,” “an,” and “the” include plural reference. Thus, for example, a reference to “a protein” includes a plurality of protein molecules.

Generally, nomenclatures used in connection with cell and tissue culture, molecular biology, immunology, microbiology, genetics, and protein and nucleic acid chemistry and hybridization described herein are those well-known and commonly used in the art. The methods and techniques provided herein are generally performed according to conventional methods well-known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery and treatment of patients.

### Definitions

That the disclosure may be more readily understood, select terms are defined below.

The term “antibody”, unless indicated otherwise, is used to refer to entire antibodies as well as antigen-binding fragments of such antibodies. For example, the term encompasses four-chain IgG molecules, as well as antibody fragments.

As used herein, the term “antibody fragments” refers to portions of an intact full-length antibody, for example, as further described below. Antibodies may be of any class, such as IgG, IgA or IgM; and of any subclass, such as IgG1, IgG3 or IgG4. Different classes and subclasses of immunoglobulin have different properties, which may be advantageous in different applications. For example, lgG4 have reduced binding to Fc receptors.

As used herein, the term “VH” refers to the variable region of the heavy chain of an antibody.

As used herein, the term “VL” refers to the variable region of the light chain of an antibody.

Naturally occurring immunoglobulins have a common core structure in which two identical light chains (about 24 kD) and two identical heavy chains (about 55 or 70 kD) form a tetramer. The amino-terminal portion of each chain is known as the variable (V) region and can be distinguished from the more conserved constant (C) regions of the remainder of each chain. Within the variable region of the light chain (i.e., the VL domain) is a C-terminal portion known as the J region. Within the variable region of the heavy chain (i.e., the VH domain), there is a D region in addition to the J region. Most of the amino acid sequence variation in immunoglobulins is confined to three separate locations in the V regions known as hypervariable regions or complementarity determining regions (CDRs) which are directly involved in antigen binding. Proceeding from the amino terminus, these regions are designated CDR1, CDR2 and CDR3, respectively. The CDRs are held in place by more conserved framework regions (FRs). Proceeding from the amino-terminus, these regions are designated FR1, FR2, FRS and FR4, respectively. The locations of CDR and FR regions and a numbering system have been defined by Kabat et al. (Kabat, E. A. et al., Sequences of Proteins of Immunological Interest, Fifth Edition, U.S. Department of Health and Human Services, U.S. Government Printing Office (1991)).

Human acceptor frameworks according to the described embodiments are set forth in SEQ ID NOs: 13-16 and 17, which may be used to as the frameworks for the humanized 2C7 antibodies described herein. Constant regions may be obtained from any human antibody constant regions. For example, the amino acid sequence of the human IgG1 CH1, CH2, and CH3 constant regions are set forth in UniProt Accession No. P01857. Human antibody framework and constant regions may be derived from sequence databases. For example, immunoglobulin sequences are available in the IMGT/LIGM database (Giudicelli et al. (2006) Nucleic Acids Res. 34:(suppl. 1):D781-D784) or VBase (vbase.mrc-cpe.cam.ac.uk).

Variable region genes of the humanized antibodies according to the described embodiments may be cloned into expression vectors in frame with constant region genes to express heavy and light immunoglobulin chains. Suitable vectors which may be used are well known in the art, and include any molecule capable of replicating in a host cell organism, into which the variable region genes of the humanized antibodies may be inserted. For example, the humanized VL and VH regions may be cloned into pMAZ-IGL and pMAZ-IGH expression vectors, respectively (Mazor Y, Barnea I, Keydar I, et al. Antibody internalization studied using a novel IgG binding toxin fusion. J Immunol Methods. 2007; 321:41-59). In an embodiment, the kappa constant region of pMAZ-IGL may be replaced with the lambda constant region sequence to express humanized antibodies comprising lambda light chains.

As used herein, the term “host cell” refers to any suitable host cell organism which may be used to express the humanized antibodies described herein. Suitable host cells include prokaryotic (e.g., E. coli) and eukaryotic (e.g., yeast, plant, insect, and mammalian) cells. In an embodiment, a suitable host cell is a eukaryotic cell. In a certain embodiment, the eukaryotic host cell is a mammalian cell. Non-limiting examples of mammalian host cells include HEK cells and CHO cells. In certain exemplary embodiments, the mammalian host cell is a CHO cell. CHO cells, which are suitable to express the humanized antibodies described herein, are known in the art and are available from a variety of commercial suppliers, e.g., American Type Culture Collection (ATCC, Manassas, VA, USA), PerkinElmer (Waltham, MA, USA), ThermoFisher Scientific (Waltham, MA, USA), Lonza (Basel, CH), as well as others.

As used herein, the term “humanized antibody” refers to an antibody which is composed of a human antibody framework, into which have been grafted complementarity CDRs from a non-human antibody. Changes in the human acceptor framework may also be made. Procedures for the design and production of humanized antibodies are well known in the art, and have been described, for example, in Cabilly et al., U.S. Pat. No. 4,816,567; Cabilly et al., European Patent Application 0 125 023; Boss et al., U.S. Pat. No. 4,816,397; Boss et al., European Patent Application 0 120 694; Neuberger, M.S. et al., WO 86/01533; Neuberger, M. S. et al., European Patent Application 0 194 276 B1; Winter, U.S. Pat. No. 5,225,539; Winter, European Patent Application 0 239 400; Padlan, E. A. et al., European Patent Application 0 519 596. Further details on antibodies, humanized antibodies, human engineered antibodies, and methods for their preparation can be found in Kontermann, R. and Dubel, S. eds. (2001, 2010) Antibody Engineering, 2nd ed., Springer-Verlag, New York, NY.

CDR-grafted antibodies comprise heavy and light chain variable region sequences from a human antibody wherein one or more of the CDR regions of VH and/or VL are replaced with CDR sequences of murine antibodies. A framework sequence from any human antibody may serve as the template for CDR grafting. However, straight chain replacement onto such a framework often leads to some loss of binding affinity to the antigen. The more homologous a human antibody is to the original murine antibody, the less likely the possibility that combining the murine CDRs with the human framework will introduce distortions in the CDRs that could reduce affinity. Therefore, in an embodiment, the human variable framework that is chosen to replace the murine variable framework apart from the CDRs have at least about 65%, at least about 70%, at least about 75%, at least about 80%, at least about 85%, at least about 90%, at least about 95%, about 100%, sequence identity with the murine antibody variable region framework. Methods for producing CDR-grafted antibodies are known in the art and described in detail along with humanization of such CDR-grafted antibodies in the Examples (see also, EP Patent No. EP 0 239 400; PCT Publication No. WO 91/09967; U.S. Pat. Nos. 5,225,539; 5,530,101; and 5,585,089); veneering or resurfacing (EP Patent Nos. EP 0 592 106 and EP 0 519 596; Padlan (1991) Mol. Immunol. 28(4/5):489-498; Studnicka et al. (1994) Protein Eng. 7(6):805-814; Roguska et al. (1994) Proc. Natl. Acad. Sci. USA 91:969-973), and chain shuffling (U.S. Pat. No. 5,565,352).

Specificity, in the context of the antibodies described herein, means that the claimed antibody is capable of selectively binding its defined cognate antigen, e.g., the 2C7 epitope of Neisseria gonorrhoeae.

As used herein, the term “2C7 epitope” refers to a highly conserved oligosaccharide structure, a part of lipooligosaccharide (LOS), on Neisseria gonorrhoeae. LOS is a critical component of the outer membrane of Neisseria gonorrhoeae. The 2C7 epitope is expressed by 94% of gonococci in the human genital tract. The structure of the 2C7 epitope requires substitution of lactose on both Heptose I and Heptose II of the LOS structure (Gulati, S., et al., PLoS Pathogens 2013 e1003559). The 2C7 epitope is known to be critical for gonococcal infection and antibodies against the 2C7 epitope are known to cause bacterial killing (Gulati, S., et al., PLoS Pathogens 2013 e1003559). The humanized antibodies disclosed herein have binding specificity for the 2C7 epitope.

As used herein, the term “Fc region” refers to the Fc region of the humanized antibody. The Fc-domain of an immunoglobulin is defined as the fragment of an antibody which would be typically generated after digestion of an antibody with papain, and which includes the two CH2-CH3 regions of an immunoglobulin and a connecting region, e.g., a hinge region. Unless otherwise specified herein, numbering of amino acid residues in the Fc region is according to the EU numbering system, also called the EU index, as described in Kabat, et al., Sequences of Proteins of Immunological Interest, 5th Ed. Public Health Service, National Institutes of Health, Bethesda, MD (1991).

As used herein, the term “complement activation” refers to the activation of the classical complement pathway, which is triggered by the binding of complement component C1q to an antibody bound to its antigen. The antibody bound to its antigen is to be understood as happening both in vivo and in vitro in the context described herein. C1q binding can be evaluated, for example, by using immobilized antibody of an artificial surface. The binding of C1q to an antibody oligomer is to be understood herein as a multivalent interaction resulting in high avidity binding.

C1q is the first protein in the early events of the classical complement cascade that involves a series of cleavage reactions that culminate in the formation of an enzymatic activity called C3 convertase, which cleaves complement component C3 into C3b and C3a. C3b binds covalently to C5 on the membrane to form C5b that in turn triggers the late events of complement activation in which terminal complement components C5b, C6, C7, C8 and C9 assemble into the membrane attack complex (MAC). The complement cascade results in the creation of pores due to which causes cell lysis, also known as complement-dependent cytotoxicity (CDC). Complement activation can be evaluated by using C1q efficacy, CDC kinetics, CDC assays (as described in WO 2014/108198, which is incorporated herein by reference), or by the method cellular deposition of C3b and C4b described in Beurskens et al Apr. 1, 2012 vol. 188 no. 7 3532-3541.

The term “complement-dependent cytotoxicity” (“CDC”), as used herein, is intended to refer to the process of antibody-mediated complement activation leading to lysis of the antibody bound to its target on a cell or bacterium as a result of pores in the membrane that are created by MAC assembly. CDC can be evaluated by in vitro assay such as a CDC assay in which normal human serum is used as a complement source, as described in WO 2014/108198, which is incorporated herein by reference, or in a C1q efficacy assay, as described in WO 2014/108198, in which normal human serum has been limited in C1q.

The term “antibody-dependent cell-mediated cytotoxicity” (“ADCC”) as used herein, is intended to refer to a mechanism of killing of antibody-coated target cells or bacteria by cells expressing Fc receptors that recognize the constant region of the bound antibody. ADCC can be determined using methods such as, e.g., the ADCC assay described in WO 2014/108198, which is incorporated herein by reference.

The term “antibody-dependent cellular phagocytosis” (“ADCP”) as used herein is intended to refer to a mechanism of elimination of antibody-coated target cells or bacteria by internalization by phagocytes. The internalized antibody-coated target cells or bacteria are contained in a vesicle called a phagosome, which then fuses with one or more lysosomes to form a phagolysosome. ADCP may be evaluated by using an in vitro cytotoxicity assay with macrophages as effector cells and video microscopy as described by van Bij et al. in Journal of Hepatology Volume 53, Issue 4, October 2010, Pages 677-685, or as described in WO 2014/108198, which is incorporated herein by reference.

The term “complement-dependent cellular cytotoxicity” (“CDCC”) as used herein is intended to refer to a mechanism of killing of target cells or bacteria by cells expressing complement receptors that recognize complement 3 (C3) cleavage products that are covalently bound to the target cells or bacteria as a result of antibody-mediated complement activation. CDCC may be evaluated in a similar manner as described for ADCC.

As used herein, the term “effector cell” refers to an immune cell which is involved in the effector phase of an immune response, as opposed to the cognitive and activation phases of an immune response. Exemplary immune cells include a cell of a myeloid or lymphoid origin, for instance lymphocytes (such as B cells and T cells including cytolytic T cells (CTLs)), killer cells, natural killer cells, macrophages, monocytes, eosinophils, polymorphonuclear cells, such as neutrophils, granulocytes, mast cells, and basophils. Some effector cells express Fc receptors (FcRs) or complement receptors and carry out specific immune functions. In some embodiments, an effector cell such as, e.g., a natural killer cell, is capable of inducing ADCC. For example, monocytes, macrophages, neutrophils, dendritic cells and Kupffer cells which express FcRs, are involved in specific killing of target cells and presenting antigens to other components of the immune system, or binding to cells that present antigens. In some embodiments the ADCC can be further enhanced by antibody driven classical complement activation resulting in the deposition of activated C3 fragments on the target cell. C3 cleavage products are ligands to complement receptors (CRs), such as CR3, expressed on myeloid cells. The recognition of complement fragments by CRs on effector cells may promote enhanced Fc receptor-mediated ADCC. In some embodiments antibody driven classical complement activation leads to C3 fragments on the target cell. These C3 cleavage products may promote direct complement-dependent cellular cytotoxicity (CDCC). In some embodiments, an effector cell may phagocytose a target antigen, target particle, or target cell. The expression of a particular FcR or complement receptor on an effector cell may be regulated by humoral factors such as cytokines. For example, expression of FcyRI has been found to be up-regulated by interferon y (IFNy) and/or G-CSF. This enhanced expression increases the cytotoxic activity of FcyRI-bearing cells against targets. An effector cell can phagocytose a target antigen or phagocytose or lyse a target cell. In some embodiments antibody driven classical complement activation leads to C3 fragments on the target cell. These C3 cleavage products may promote direct phagocytoses by effector cells or indirectly by enhancing antibody mediated phagocytosis.

In the context of the present invention, a substitution or mutation in a variant is indicated as: original amino acid-position-substituted amino acid. To indicate each amino acid, the three-letter code, or one letter code, are used, including the codes Xaa and X. Accordingly, the notation “R435H” or “Arg435His” means that the variant comprises a substitution of Arginine with Histidine in the variant amino acid position corresponding to the amino acid in position 435 in the parent antibody. Where a position as such is not present in an antibody, but the variant comprises an insertion of an amino acid, for example: position-substituted amino acid; the notation, e.g., “448E” is used. Such notation is particular relevant in connection with modification(s) in a series of homologous polypeptides or antibodies. Similarly when the identity of the substitution amino acid residues(s) is immaterial: original amino acid-position; or “R435”. For a modification where the original amino acid(s) and/or substituted amino acid(s) may comprise more than one, but not all amino acid(s), the substitution of Glutamic acid for Tryptophan, Lysine or Tryptophan in position 435: “Arg435Thr,Lys,Trp” or “R435T,K,W” or “R435T/K/W” or “R435 to T, K or W” may be used interchangeably in the context of the invention. Furthermore, the term “a substitution” embraces a substitution into any one of the other nineteen natural amino acids, or into other amino acids, such as non-natural amino acids. For example, a substitution of amino acid E in position 435 includes each of the following substitutions: 435A, 435C, 435D, 435G, 435H, 435F, 4351, 435K, 435L, 435M, 435N, 435Q, 435R, 435S, 435T, 435V, 435W, and 435Y. This is, by the way, equivalent to the designation 435X, wherein the X designates any amino acid. These substitutions can also be designated 435S435A, 435S435C, etc., or 435S435A, C, etc., or 435S435A/C/etc. The same applies to analogy to each and every position mentioned herein, to specifically include herein any one of such substitutions.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting.

### Antibodies

The invention encompasses humanized antibodies that bind a specific epitope of Neisseria gonorrhoeae (e.g., 2C7) for treating Neisseria gonorrhoeae infection. In one aspect, the humanized antibodies disclosed herein are a humanized version of the murine monoclonal 2C7 antibody set forth in SEQ ID NO: 1 (VH) and SEQ ID NO: 2 (VL), shown in Table 1 below.

TABLE 1
Sequence of Mouse Monoclonal 2C7
Antibody Variable Regions

| Variable | Sequence | |
|---|---|---|
| Region | Identifier | Sequence |
| Variable | SEQ ID NO: 1 | EVQLQQSGPELVKPGSSVKISC |
| Heavy | | KGSGYTFTDYNMEWVKQSHGKS |
| (VH) | | LEWIGVINPNNRFTSYNQNFRG |
| | | KATLTVDKSSSTAYMDLRSLTS |
| | | EDSAVYFCAGSRWYQYDYWGQG |
| | | TTLTVSS |
| Variable | SEQ ID NO: 7 | QVVVTQESALTTSPGETVTLTC |
| Light | | RSSTGAVTTSNYANWVQEKPDH |
| (VL) | | LFTGLIGGINNRAPGVPARFSG |
| | | SLIGDKAALTITGAQTEDEAIY |
| | | FCALWYSNHWVFGGGTKLTVL |

In another aspect, provided herein is a humanized antibody that binds the 2C7 epitope of Neisseria gonorrhoeae antigen, wherein the antibody comprises a variable heavy (VH) domain, a variable light (VL) domain, and a human Fc region, wherein the VH domain comprises an HCDR-1, an HCDR-2, and an HCDR3 selected from the amino acid sequence set forth in SEQ ID NO: 1 and a VH human acceptor framework comprising an amino acid sequence selected from the group consisting of SEQ ID NOs: 13-16 (Table 2), and wherein the VL domain comprises an LCDR-1, LCDR-2, and an LCDR-3 selected from the amino acid sequence set forth in SEQ ID NO: 7 and a VL human acceptor framework comprising an amino acid sequence selected from the amino acid sequence set forth in SEQ ID NO: 17 (Table 3).

In another aspect, provided herein is a humanized antibody that binds the 2C7 epitope of Neisseria gonorrhoeae antigen, wherein the antibody comprises a variable heavy (VH) domain, a variable light (VL) domain, and a human Fc region, wherein the VH domain comprises an HCDR-1, an HCDR-2, and an HCDR3 selected from the amino acid sequence set forth in SEQ ID NO: 1 and a VH human acceptor framework comprising an amino acid sequence with at least about 90% identity to about 99% identity to an amino acid sequence selected from the group consisting of SEQ ID NOs: 13-16, and wherein the VL domain comprises an LCDR-1, LCDR-2, and an LCDR-3 selected from the amino acid sequence set forth in SEQ ID NO: 7 and a VL human acceptor framework comprising an amino acid sequence with at least about 90% identity to about 99% identity to an amino acid sequence selected from the amino acid sequence set forth in SEQ ID NO: 17.

TABLE 2
Sequence of Humanized 2C7 Antibody
Variable Heavy Regions

| Sequence | |
|---|---|
| Identifier | Sequence |
| SEQ ID | QVQLVQSGAEVKKPGASVKVSCKASGYTFTDYNMEWV |
| NO: 2 | RQAPGQGLEWMGVINPNNRFTSYNQNFRGRVTMTRDT |
| (VH1) | STSTVYMELSSLRSEDTAVYYCARSRWYQYDYWGQGT |
| | LVTVSS |
| SEQ ID | QVQLVQSGAEVKKPGASVKVSCKASGYTFTDYNMEWV |
| NO: 3 | RQAPGQRLEWMGVINPNNRFTSYNQNFRGRVTITRDT |
| (VH2) | SASTAYMELSSLRSEDTAVYYCARSRWYQYDYWGQGT |
| | LVTVSS |
| SEQ ID | QVQLVQSGAEVKKPGSSVKVSCKASGYTFTDYNMEWV |
| NO: 4 | RQAPGQGLEWMGVINPNNRFTSYNQNFRGRVTITADK |
| (VH3) | STSTAYMELSSLRSEDTAVYYCARSRWYQYDYWGQGT |
| | LVTVSS |
| SEQ ID | QVQLVQSGSELKKPGASVKVSCKASGYTFTDYNMEWV |
| NO: 5 | RQAPGQGLEWMGVINPNNRFTSYNQNFRGRFVFSLDT |
| (VH4) | SVSTAYLQISSLKAEDTAVYYCARSRWYQYDYWGKGT |
| | TVTVSS |
| SEQ ID | EVQLVQSGAEVKKPGESLKISCKGSGYTFTDYNMEWV |
| NO: 6 | RQMPGKGLEWMGVINPNNRFTSYNQNFRGQVTISADK |
| (VH5) | SISTAYLQWSSLKASDTAMYYCARSRWYQYDYWGQGT |
| | LVTVSS |
| SEQ ID | EVQLVQSGAEVKKPGESLKISCKGSGYTFTDYNMEWV |
| No: 13 | RQMPGKGLEWMGVINPNNRFTSYNQNFRGQVTISADK |
| (VH5 or | SISTAYLQWSSLKASDTAMYYCAGSRWYQYDYWGQGT |
| ID No: 6 | LVTVSS |
| with 1 | |
| back- | |
| mutation) | |
| SEQ ID | EVQLVQSGAEVKKPGESLKISCKGSGYTFTDYNMEWV |
| No: 14 | RQMPGKGLEWIGVINPNNRFTSYNQNFRGQVTISADK |
| (SEQ ID | SISTAYLQWSSLKASDTAMYFCAGSRWYQYDYWGQGT |
| No: 6 | LVTVSS |
| with 3 | |
| back- | |
| mutations) | |
| SEQ ID | EVQLVQSGAEVKKPGESLKISCKGSGYTFTDYNMEWV |
| No: 15 | RQMPGKGLEWIGVINPNNRFTSYNQNFRGQVTISADK |
| (SEQ ID | SISTAYLQWSSLKASDTAMYYCAGSRWYQYDYWGQGT |
| No: 6 | LVTVSS |
| with 2 | |
| back- | |
| mutations) | |
| SEQ ID | EVQLVQSGAEVKKPGESLKISCKGSGYTFTDYNMEWV |
| No: 16 | RQMPGKGLEWMGVINPNNRFTSYNQNFRGQVTISADK |
| (SEQ ID | SISTAYLQWSSLKASDTAMYFCAGSRWYQYDYWGQGT |
| No: 6 | LVTVSS |
| with 2 | |
| back- | |
| mutations) | |

TABLE 3
Sequence of Humanized 2C7 Antibody
Variable Light Regions

| Sequence | |
|---|---|
| Identifier | Sequence |
| SEQ ID NO: 8 | QAVVTQEPSLTVRSSTGAVTTSNYANWFQ |
| | QKPGQAPRTLIYGINNRAPWTPARFSGSL |
| | LGGKAALTLSGAQPEDEAEYYCALWYSNH |
| | WVFGGGTKLTVL |
| SEQ ID NO: 9 | QTVVTQEPSLTVSPGGTVTLTCRSSTGAV |
| | TTSNYANWFQQKPGQAPRALIYGINNRAP |
| | WTPARFSGSLLGGKAALTLSGVQPEDEAE |
| | YYCALWYSNHWVFGTGTKVTVL |
| SEQ ID NO: 10 | QTVVTQEPSFSVSPGGTVTLTCRSSTGAV |
| | TTSNYANWYQQTPGQAPRTLIYGINNRAP |
| | GVPDRFSGSILGNKAALTITGAQADDESD |
| | YYCALWYSNHWVFGGGTKLTVL |
| SEQ ID NO: 11 | EIVMTQSPATLSVSPGERATLSCRSSTGA |
| | VTTSNYANWYQQKPGQAPRLLIYGINNRA |
| | PGIPARFSGSGSGTEFTLTISSLQSEDFA |
| | VYYCALWYSNHWVFGGGTKVEIK |
| SEQ ID NO: 12 | QSVLTQPPSASGTPGQRVTISCRSSTGAV |
| | TTSNYANWYQQLPGTAPKLLIYGINNRAP |
| | GVPDRFSGSKSGTSASLAISGLQSEDEAD |
| | YYCALWYSNHWVFGGGTKLTVLG |
| SEQ ID No: 17 | QTVVTQEPSLTVSPGGTVTLTCRSSTGAV |
| (SEQ ID No: 9 | TTSNYANWFQQKPGQAPRGLIGGINNRAP |
| with 3 back- | GTPARFSGSLLGGKAALTLSGVQPEDEAE |
| mutations) | YYCALWYSNHWVFGTGTKVTVL |

In an embodiment of the humanized antibody, the VH domain comprises the amino acid sequence of SEQ ID NO: 13 and the VL domain comprises the amino acid sequence of SEQ ID NO: 17.

In an embodiment of the humanized antibody, the VH domain comprises the amino acid sequence of SEQ ID NO: 2 and the VL domain comprises the amino acid sequence of SEQ ID NO: 9.

In an embodiment of the humanized antibody, the VH domain comprises the amino acid sequence of SEQ ID NO: 14 and the VL domain comprises the amino acid sequence of SEQ ID NO: 17.

In an embodiment of the humanized antibody, the VH domain comprises the amino acid sequence of SEQ ID NO: 15 and the VL domain comprises the amino acid sequence of SEQ ID NO: 17.

In an embodiment of the humanized antibody, the VH domain comprises the amino acid sequence of SEQ ID NO: 16 and the VL domain comprises the amino acid sequence of SEQ ID NO: 17.

In any use according to the disclosed invention, the humanized 2C7 antibody without any additional mutations is termed a “parent humanized antibody.” Thus, the uses herein provides for any variants of such parent humanized antibodies.

In one embodiment the parent humanized antibody may be a parent antibody comprising an Fc domain, an immunoglobulin, and an antigen-binding region. Introducing a mutation to a humanized antibody according to a method or use of the present invention results in a variant humanized antibody (which may also be referred to as a “variant” herein). Thus, the method(s) of the present invention may be performed so as to obtain any variant or variant humanized antibody as described herein.

In an embodiment, a variant humanized antibody obtained from a method or use of the present invention has an increased CDC compared to the parent humanized antibody. Typically, the effect of a humanized antibody on an effector function may be determined by the EC₅₀ value, which is the concentration of the antibody necessary to obtain half the value of the maximal lysis.

Maximal lysis is the lysis obtained when a saturating amount of the humanized antibody is used in which saturating is intended to refer to the amount of humanized antibody at which all targets for the antibody are bound by antibody.

In one embodiment the effector function is Fc-receptor binding, e.g., including Fc-gamma receptor-binding. In one embodiment the effector function is Fc-containing antibody internalization. In one embodiment the effector function is a combination of complement CDC and ADCC.

As used herein, the term “C1q-binding”, when used in the context of a variant or antibody of a parent humanized antibody includes any mechanism of the first component on the classical pathway of complement activation mediated by binding of the variant or antibody to host tissues or factors, including various cells of the immune system (such as effector cells). C1q-binding of an antibody can be evaluated using an ELISA (such as, e.g., C1q binding ELISA), or the C1q efficacy can be evaluated by a CDC.

In an embodiment of the humanized antibodies of the present invention, one or more mutations in the IgG3 Fc region leads to increased effector or Fc functions. In an embodiment of the humanized antibodies, one or more mutations in the IgG3 Fc region leads to increased CDC against Neisseria gonorrhoeae.

In an embodiment of the humanized antibodies of the present invention, the one or more mutations in the IgG3 Fc is R435H confers the ability to bind to protein A. In an embodiment of the humanized antibodies, one or more mutations in the IgG3 Fc region leads to increased half-life.

The term “increasing CDC,” “improving CDC,” “increasing an effector function,” or “improving an effector function,” refers in the context of the present invention that there is a decrease in the EC₅₀ value of the variant humanized antibody compared to the parent humanized antibody. The decrease in the EC₅₀ value may, e.g., be at least or about 2-fold, such as at least or about 3-fold, or at least or about 5-fold, or at least or about 10-fold. Alternatively, “increasing CDC,” “improving CDC,” “increasing an effector function,” or “improving an effector function,” means that there is an increase in the maximal amount of cells lysed (where the total amount of cells is set at 100%) by, e.g., from 10% to 100% of all cells, such as by about 10%, about 20%, about 30%, about 40%, about 50%, about 60%, about 70%, about 80%, about 90%, and about 100% under conditions where the humanized antibody lyses less than 100% of all cells.

A variant could be tested for increased or improved effector function by cloning the variable domain into the variant and test its efficacy in CDC assays.

Using a humanized antibody disclosed herein and gonococci, an increase in efficacy would be defined by a more than 2-fold lower EC₅₀ than the EC₅₀ of the humanized antibody under the studied condition, such as about 2-fold, about 3-fold, about 5-fold, about 10-fold or a more than 10-fold lower EC₅₀ value, the concentration at which half-maximal lysis is observed.

An increase in CDC could also be defined by an increase in the maximal lysis ranging from 10% to 100% of all cells, such as by about 10%, about 20%, about 30%, about 40%, about 50%, about 60%, about 70%, about 80%, about 90%, and about 100%.

The inventors of the present invention found that using one or more previously disclosed Fc mutation (WO 2014/108198, which is incorporated herein by reference) in a humanized antibody of the invention, resulted in increased bactericidal activity against Neisseria gonorrhoeae infections, as compared to the murine parent antibody. Without being bound by theory, it is believed that by substituting one or more amino acid(s) from the above-mentioned group of positions, antibody oligomerization is stimulated. The Fc regions of the antibodies bind one or more Fc ligand with higher avidity, thereby enabling different effector functions, e.g., CDC, ADCC, and ADCP. Fc ligands include but are not limited to FcγRs, FcγRs, FcγRs, FcRn, C1q, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (Davis et al., 2002, Immunological Reviews 190:123-136, hereby entirely incorporated by reference). Fc ligands may include undiscovered molecules that bind Fc.

The inventors of the present invention found that using IgG3 where R (Arg) at position 435 (Eu numbering) is replaced with H (His), confers both a half-life comparable to human IgG1 and the ability to bind to protein A.

In one embodiment, at least one other effector function of the antibody, such as C1q-binding, complement activation, ADCC, Fc-gamma receptor-binding, ADCP, CDCC, complement-enhanced cytotoxicity, ADCP, internalization, apoptosis, and/or binding to complement receptor of a humanized antibody, is also increased.

In one embodiment, the CDC of the parent antibody is increased when the parent antibody is bound to its antigen on an antigen-expressing bacterium.

In one embodiment of the methods and/or uses of the present invention the parent humanized antibody, may contain other mutations than those of the present invention which have been found to affect an effector function. Such other mutations may be introduced at the same time as the mutations of the present invention which affect an effector function or they may be introduced sequentially, the methods or uses of the present invention are not limited to either simultaneous or sequential introduction of mutations.

In one embodiment, the one or more mutation(s) is one mutation, i.e. no more than one mutation is introduced to the parent humanized antibody. In another embodiment, the method or use according to the present invention comprises introducing a mutation in at least two, such as two, three, four, five, or more of amino acids.

Any of the combinations of mutations described herein may be introduced according to a method of the present invention.

In the methods or uses according to the present invention, CDC is increased when the antibody is bound to its antigen. Without being bound to any theory it is believed that CDC is increased when the antibody is bound to its antigen, wherein the antigen is on gonococci.

In a main aspect the present invention relates to a method of inducing CDC against Neisseria gonorrhoeae expressing the 2C7 epitope to which a parent humanized antibody comprising an Fc-domain of an immunoglobulin (IgG3) and the humanized binding region: (i) provides a humanized antibody which has been mutated according to any one of the embodiments disclosed herein; and (ii) the mutated humanized antibody of step (ii) binds to (—or contacts) Neisseria gonorrhoeae expressing the 2C7 epitope in the presence of human complement or an effector cell.

In a further embodiment the method also induces ADCC.

In yet a further embodiment the method also induces Fc-containing antibody internalization.

In one embodiment, the contacting step (ii) takes place in vitro.

In one embodiment, the contacting step (ii) takes place in vivo.

In another embodiment, step (ii) comprises administering the variants to a subject infected with Neisseria gonorrhoeae.

In another embodiment, step (ii) comprises administering the variants to a subject infected with antibiotic resistant Neisseria gonorrhoeae.

Without being bound by any theory, it is believed that the enhancement of CDC can be restricted to target cells that express two specific targets/antigens simultaneously provided that the first and second antibody bind epitopes found on the same cell, thereby exploiting the combined expression of targets to improve selectivity of enhanced CDC induction.

### Methods of Treating Neisseria gonorrhoeae

In yet another aspect, provided herein are methods of treating Neisseria gonorrhoeae infection in a subject in need thereof comprising administering to the subject an antibody disclosed herein or the pharmaceutical compositions disclosed herein.

In an embodiment of the methods, the administered antibody or pharmaceutical composition improves clearance of the Neisseria gonorrhoeae infection.

In an embodiment of the methods, the administered antibody or pharmaceutical composition protects the subject from a subsequent Neisseria gonorrhoeae infection.

In another embodiment of the methods, the administered antibody or pharmaceutical composition improves clearance of the Neisseria gonorrhoeae infection and protects the subject from a subsequent Neisseria gonorrhoeae infection.

In another embodiment of the methods, the Neisseria gonorrhoeae infection is resistant to antibiotics.

In yet another embodiment of the methods, the subject is a human.

For therapeutic and prophylactic uses, the antibodies and antibody fragments of the present invention may be formulated as a pharmaceutical composition comprising an immunotherapeutically or immunoprophylactically effective amount of the antibody or antibody fragment admixed with a pharmaceutically acceptable carrier, the amount being effective to significantly kill the infecting organism in the presence of complement, or to opsonize the infecting organism to permit phagocytic killing by host PMNs.

Exemplary pharmaceutical compositions of this invention will be suspended in a sterile solution for therapeutic uses. The pharmaceutical compositions may alternatively be formulated to control release of the active ingredients or to prolong their presence in a patient's system. Numerous suitable drug delivery systems are known and include, e.g., implantable drug release systems, hydrogels, hydroxymethylcellulose, microcapsules, liposomes, microemulsions, microspheres, and the like.

The pharmaceutical compositions of this invention may be administered by any suitable means such as orally, intranasally, subcutaneously, intramuscularly, intravenously, intra-arterially, or parenterally. In exemplary embodiments, intravenous (i.v.) or parenteral administration will be used.

It will be apparent to those of ordinary skill in the art that the immunotherapeutically effective or immunoprophylactically effective amount of antibody or fragments thereof of this invention will depend, inter alia, upon the administration schedule, the unit dose of antibody or fragment administered, whether the antibody or fragment is administered in combination with other therapeutic agents, the immune status and health of the patient, the therapeutic activity of the antibody or antibody fragment administered and the judgment of the treating physician.

The antibodies or fragments thereof according to the present invention may also be labeled and used in screening methods, diagnostic methods, or assays for detecting antibodies reactive with oligosaccharide antigens of N. gonorrhoeae in vitro or in vivo. These include, for example, enzyme-linked immunosorbent assays (ELISAs). For example, samples may be screened for the presence of antibodies reactive with oligosaccharide antigens of N. gonorrhoeae by contacting the sample with a labeled antibody of the present invention and detecting the label. Similarly, antibodies may also be prepared and used for detecting the presence of gonococcal oligosaccharide (OS) antigen present in clinical samples. Accordingly, this invention includes diagnostic kits comprising detectably labeled antibodies or fragments or antibodies or fragments of this invention, as a reagent, and complete instructions for using the reagent to detect antibodies reactive with oligosaccharide antigens of N. gonorrhoeae or the oligosaccharide antigens themselves. Detection methods according to this invention may comprise the steps of applying anti-immunoglobulin antibodies to a solid support; applying a biological sample to the solid support; removing the excess biological sample from the solid support; applying detectably labelled antibodies or fragments according to this invention to the solid support; washing the solid support and assaying for the presence of label on the solid support.

Suitable labels may be radioactive, enzymatic, fluorescent, magnetic or chemiluminescent. Radiolabeled antibodies are prepared in known ways by coupling a radioactive isotope such as ³H, ³²P, ³⁵S, ⁵⁹Fe, ¹²I, which can then be detected by gamma counter, scintillation counter, or by autoradiography. Antibodies of this invention may be suitably labeled with enzymes such as yeast alcohol dehydrogenase, horseradish peroxidase, alkaline phosphatase, and the like, then developed and detected spectrophotometrically or visually. Suitable fluorescent labels include fluorescein isothiocyanate, fluorescamine, rhodamine, and the like. Suitable chemiluminescent labels include luminol, imidazole, oxalate ester, luciferin, and the like.

### EXAMPLES

### Example 1: Colonization of Neisseria gonorrhoeae in Mouse Model

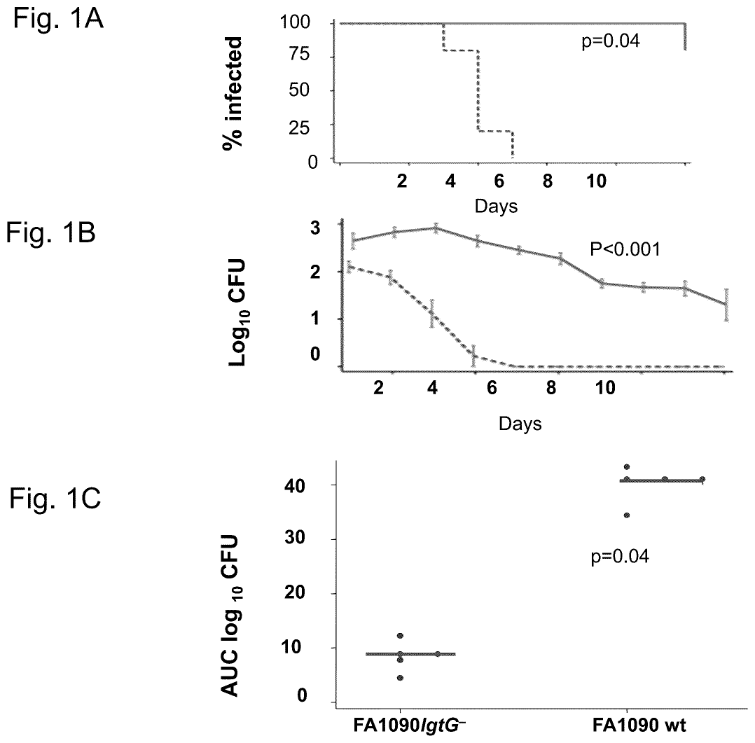

A strain of N. gonorrhoeae with a knockout of the 2C7 epitope was analyzed for infection rates in mice. The knockout (FA1090lgtG−) and wild-type (FA1090wt) N. gonorrhoeae strains were mixed in equal proportions and inoculated into non-immune mice (n=5). Clearance of the infection was analyzed. All five mice infected with the FA1090lgtG− strain cleared infection by day 7. In contrast, all the mice infected with FA1090wt strain were still infected beyond day 10 (FIG. 1A). Colonization of each strain was measured at daily intervals; colonization with FA1090lgtG− was completely absent by day 7, whereas FA1090wt maintained colonization beyond day 10 (FIG. 1B). Area under the curve analysis shows that infection with FA1090wt resulted in significantly higher bacterial burdens in non-immune mice compared to FA1090lgtG− (FIG. 1C).

### Example 2: Bactericidal Activity of Humanized Monoclonal Antibodies

Complement (C) dependent killing of N. gonorrhoeae of mouse and humanized 2C7 monoclonal antibodies were compared. Bactericidal assays were performed with an antibody concentration series or at a fixed antibody concentration. IgG-IgM depleted normal human serum (20% v/v; final concentration unless indicated otherwise) was used as a source of complement. Briefly, 2000 CFU of bacteria grown to the mid-log phase were suspended in HBSS²⁺. Monoclonal antibodies were each mixed with gonococci and serum; the final volume of all reaction mixtures was 150 μl. 25 μl aliquots of the reaction mixtures were plated onto chocolate agar plates at time 0 and 30 min. Percent (%) survival was expressed as the percent of colonies on a plate at 30 min compared to those on the plate at 0 min.

### Example 3: Design and Production of Humanized 2C7 Monoclonal Antibody Variants

### Humanized 2C7

Humanized 2C7 monoclonal antibodies described herein are variants of 2C7 mouse monoclonal antibody. The humanized 2C7 variants were designed initially to replace 2C7 mouse Fc with human IgG1 Fc; 2C7 mouse constant heavy and light domains in the Fab regions were replaced with constant heavy and light domains of human IgG1. Multiple variants of the humanized 2C7 mAb are described herein. By applying standard methods well known in the art, the CDR sequences of VH and VL chains of monoclonal antibody 2C7 (see Table 1) were grafted onto different human heavy and light chain acceptor sequences. The heavy chain and light chain variable regions of the humanized antibodies were cloned into an immunoglobulin G1 (IgG1) expression vector described previously.

### Production

Humanized 2C7 antibodies were produced using standard cell culture methods and purification techniques. CDR-grafted VL and VH sequences were cloned into pMAZ-IGL and pMAZ-IGH expression vectors, respectively (Mazor Y, Barnea I, Keydar I, et al. Antibody internalization studied using a novel IgG binding toxin fusion. J Immunol Methods. 2007; 321:41-59). The kappa constant region of pMAZ-IGL was replaced with the lambda constant sequence (murine mAb 2C7 is IgG3 lambda) to express monoclonal antibodies with lambda light chains.

Plasmids comprising each of 25 light-heavy chain combinations were used to transfect ExpiCHO-S™ Cells (ThermoFisher Scientific, Cat #A29127). Secreted antibodies were purified over protein A/G agarose and tested for their ability to: i) bind to gonococcal lipooligosaccharide (LOS) in ELISA assays and to intact bacteria by flow cytometry and ii) to mediate killing in complement-dependent bactericidal assays. Chimeric mAb 2C7 was used as a positive control.

### Example 4 Binding Activity of the Humanized 2C7 Monoclonal Antibody by ELISA

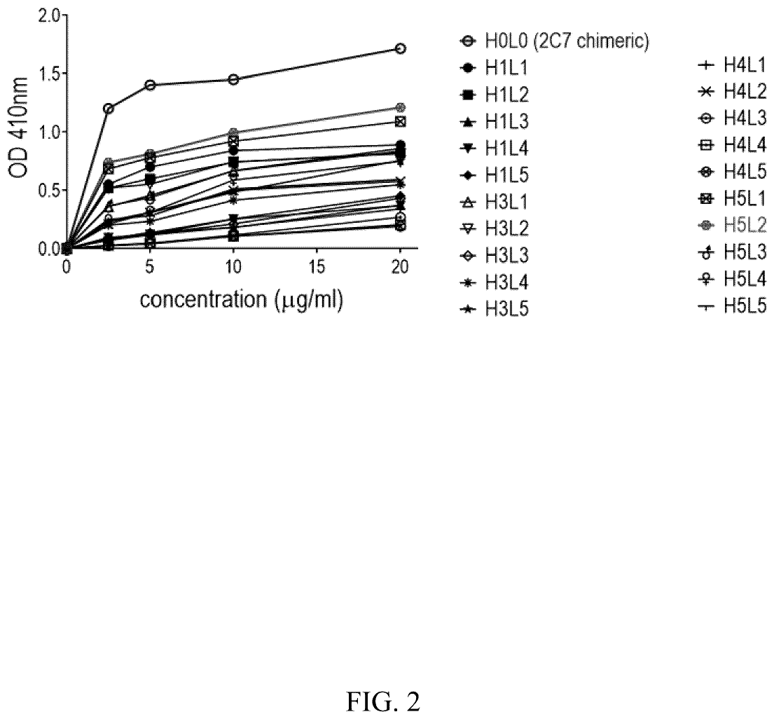

Humanized 2C7 antibodies were produced using standard cell culture methods and purification techniques. 20 humanized 2C7 monoclonal antibodies with mouse CDRs were generated. Humanized 2C7 variants were designed as follows: 2C7 mouse Fc was replaced by human IgG1 Fc and the 2C7 mouse constant heavy and light domains of the Fab regions were replaced by constant heavy and light domains of human IgG1. 20 humanized 2C7 monoclonal antibodies with mouse CDRs (H1L1, H1L2, H1L3, H1L4, H1L5, H3L1, H3L2, H3L3, H3L4, H3L5, H4L1, H4L2, H4L3, H4L4, H4L5, H5L1, H5L2, H5L3, H5L4, H5L5) were generated by combining variable domains of 5 heavy chains (VH1, VH2, VH3, VH4 and VH5) and 5 light chains (VL1, VL2, VL3, VL4 and VL5) (FIG. 2 [the VH2 construct did not express protein]).

Binding activities of the 20 humanized 2C7 monoclonal antibodies were analyzed by ELISA assays (FIG. 2). Each mAb was diluted in PBS containing 0.05% Tween 20 and dispensed into microtiter wells (Immulon 1B) coated LOS purified from N. gonorrhoeae strain 15253; reactions were caried out for 1 hour at room temperature. Antibody binding was detected with anti-human alkaline phosphatase secondary antibody and para-nitrophenylphosphate (PNPP) used as substrate.

### Example 5 Binding Activity of the Humanized 2C7 Monoclonal Antibody to intact N. gonorrhoeae by Flow Cytometry

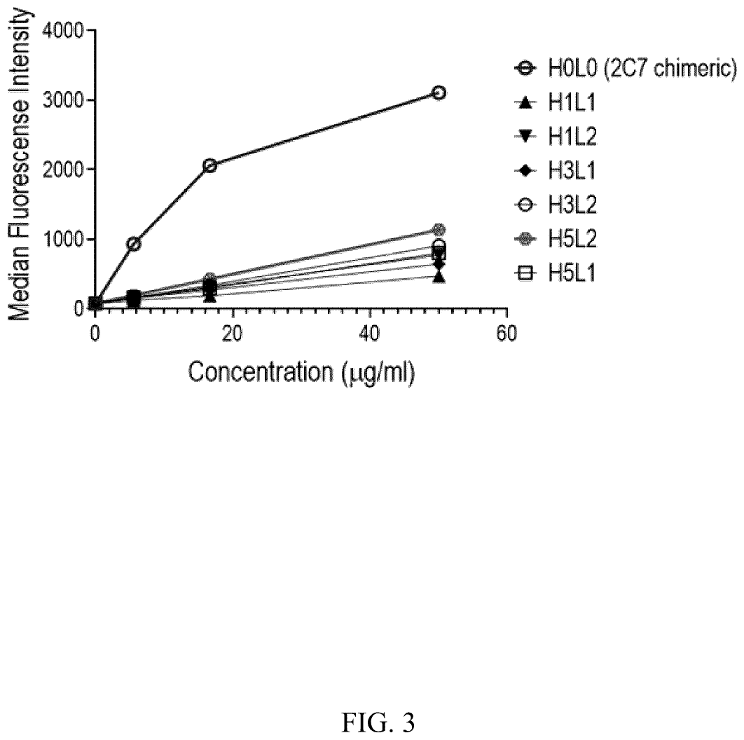

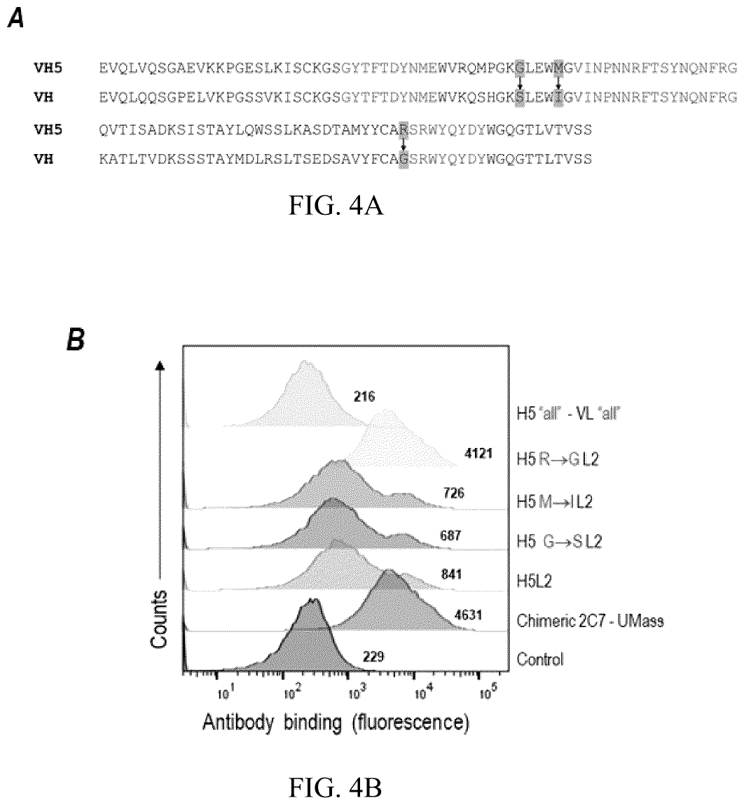

Six monoclonal antibodies among the humanized monoclonal antibodies that showed the best binding activity against N. gonorrhoeae LOS in FIG. 2 (H1L1, H1L2, H3L1, H3L2, H5L2, and H5L1) were tested for their ability to bind N. gonorrhoeae strain 15253 by flow cytometry; the VH5-VL2 combination showed the best binding to N. gonorrhoeae (FIG. 3; H5L2). H5L2 showed approximately 5.5-fold less binding than chimeric mAb 2C7 binding (FIG. 4B, labeled “Chimeric 2C7-UMass”).

### Example 6: The Effect of VH5 Back Mutations on Binding of Humanized 2C7 mAb H5L2 Variants to N. gonorrhoeae Strain 15253

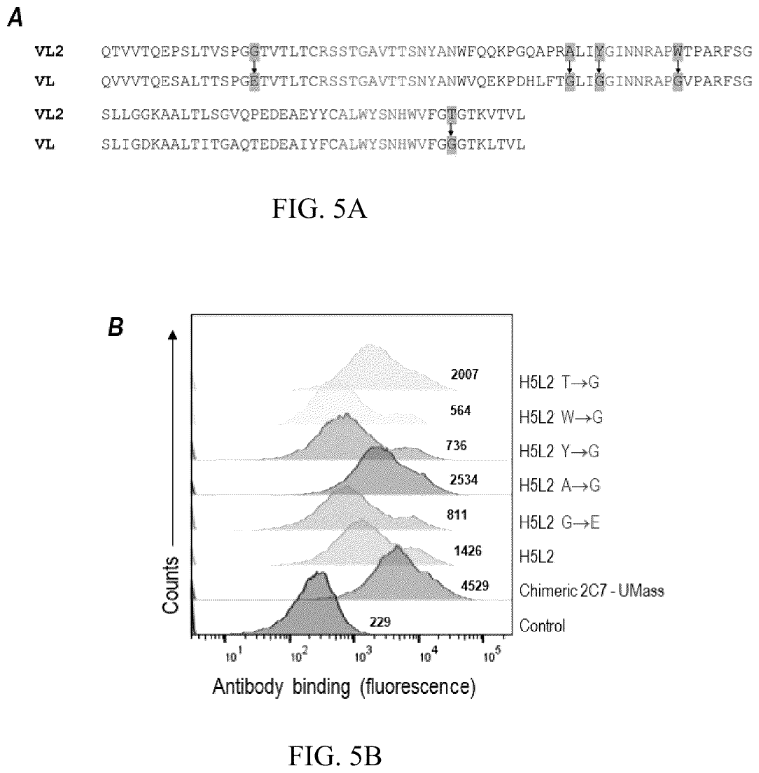

Monoclonal antibodies with three backmutations were created, H5 G→S, replacing glycine with serine, H5 M→I, replacing methionine with isoleucine, and H5 R→G, replacing arginine with glycine (FIG. 4A). All variants had the VL2 sequence. Variants with fully back-mutated VL2 sequences and with fully back-mutated VH5 were also created (FIGS. 4A and 5A); (labeled H5 “all”-VL “all” in FIG. 4B). The binding to N. gonorrhoeae strain 15253 of each of the VH5 back mutated variants was tested and compared to the H5 variant with the VL2 sequence (labeled H5L2 in FIG. 4B). Chimeric mAb 2C7, used as a positive control, showed the highest binding (FIG. 4B); variant VH5 R→G VL2 showed binding similar to chimeric 2C7 (humanized 2C7 monoclonal antibodies with the VL2 sequence were abbreviated as L2 in FIG. 4B).

### Example 7: The Effect of VL2 Back Mutations on Binding of Humanized 2C7 mAb H5L2 Variants to N. gonorrhoeae Strain 15253

Monoclonal antibodies with five VL2 back mutations were created (H5L2 T→G, replacing threonine with glycine; H5L2 W→G, replacing tryptophan with glycine; H5L2 Y→G, replacing tyrosine with glycine; H5L2 A→G, replacing alanine with glycine; and H5 G→E, replacing glycine with glutamate [FIG. 5A]). The binding to N. gonorrhoeae strain 15253 of these variants was tested and compared to the mAb 2C7 chimeric positive control (labeled Chimeric 2C7-UMass in FIG. 5B). Humanized VH5 WT in combination with VL2 back mutations A→G or T→G showed approximately half the amount of binding exhibited by the positive control (labeled Chimeric 2C7-UMass in FIG. 5B).

Example 8: The Effect of H5 and L2 Back Mutations on Binding of Humanized 2C7 mAb H5L2 variants to N. gonorrhoeae Strain 15253

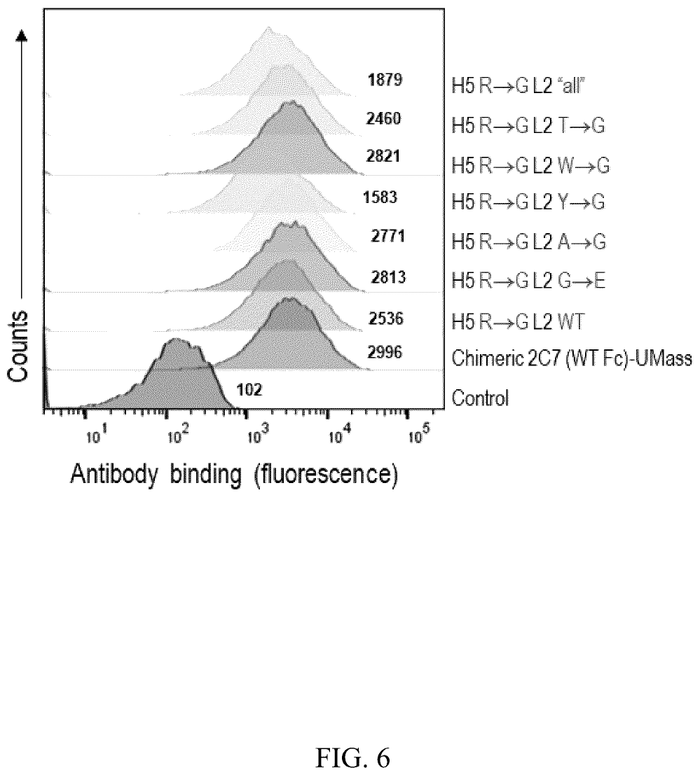

Combinations of VH5 R→G with all VL2 back mutations were generated. Binding of these variants to N. gonorrhoeae strain 15253 was tested and compared to binding by the positive control (labeled Chimeric 2C7 (WT Fc)-UMass in FIG. 6). VH5 R→G and most of the VL2 variants showed similar binding to chimeric 2C7. Variants VH5 R→G L2 Y→G and VH5 R→G L2 “all” showed slightly lower binding than the positive control (FIG. 6).

### Example 9: Bactericidal Activity of Humanized 2C7 mAb H5L2 Variants Containing the H5 R→G Variants and VL2 Y→G, A→G and W→G Back Mutations

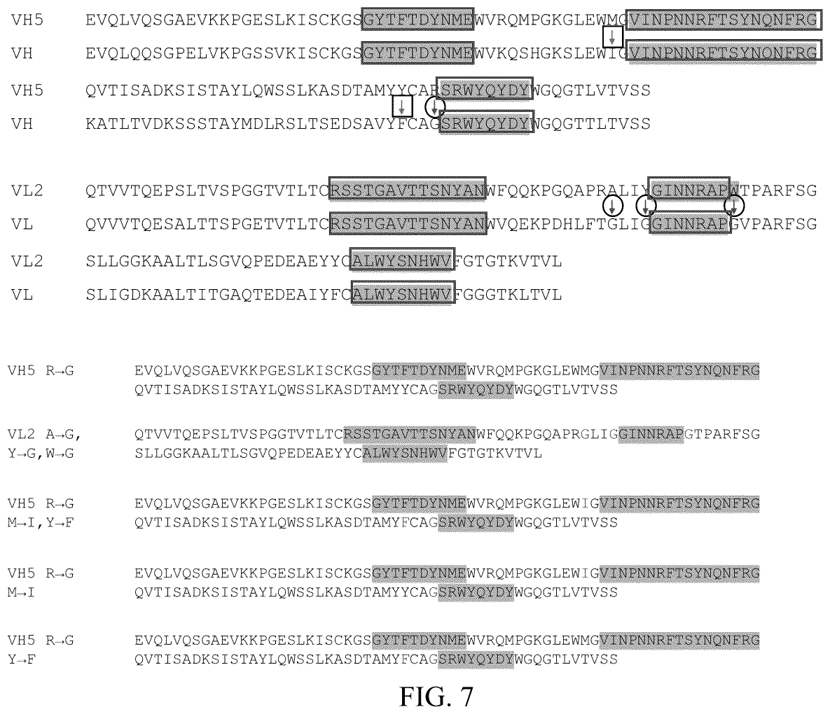

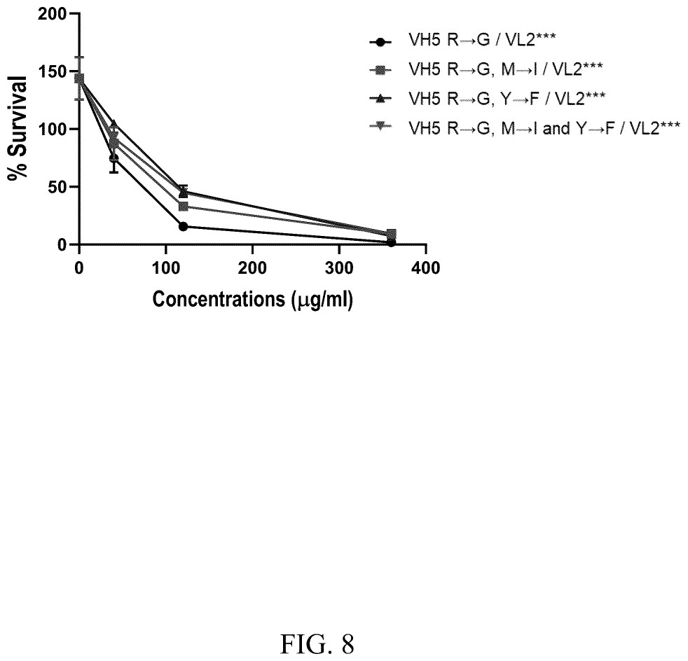

Bactericidal activity of variant VH5 R→G with VL2 Y→G was hypothesized to be attributable to the VL2 Y→G mutation because the mutation was located near CDR2. Therefore, additional mutations of VL2 near the CDR2 region were performed (A→G and W→G, FIG. 7). Complement dependent bactericidal assays showed that only H5L2Y-G and H5L2‘all’ displayed minimal killing activity with human IgG1 Fc (IC₅₀ ˜300 μg/mL). To further increase complement activation, a molecule with IgG3 Fc was constructed. Bactericidal activity of humanized 2C7 mAb H5L2 variant VH5 R→G with VL2 mutations A→G, Y→G, and W→G (human IgG3) against N. gonorrhoeae FA1090 was evaluated. Variant VH5 R→G with all 3 VL2 mutations: A→G; Y→G; and W→G (designated VL2*** in FIG. 8) demonstrated dose responsive bactericidal activity against N. gonorrhoeae FA1090 (FIG. 8). Additional mutations in VH5 R→G (M→I and Y→F) were also performed (boxed arrows, FIG. 7). Bactericidal activity of the additional mutated antibodies is shown in FIG. 8.

### Example 10: Bactericidal Activity of Humanized 2C7 mAb with Human IgG3 Fc Against N. gonorrhoeae Strains FA1090 and 15253

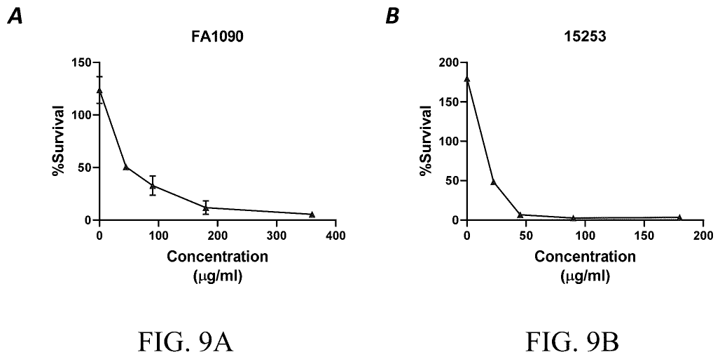

Human (h)IgG1 was replaced with hIgG3 Fc because of better complement activation by hIgG3 Fc. In addition, R (Arg) at position 435 (Eu numbering) in IgG3 (GenBank Accession number CAA67886.1) was replaced with H (His), R435H, which confers a half-life comparable to human IgG1 and the ability to bind to protein. 2C7 mAb with human IgG3 showed greater bactericidal activity against N. gonorrhoeae strains FA1090 and 15253 (FIGS. 9A-9B).

### Example 11: Efficacy of Humanized 2C7 mAb hIgG3 Fc in a Mouse Model of Gonorrhea

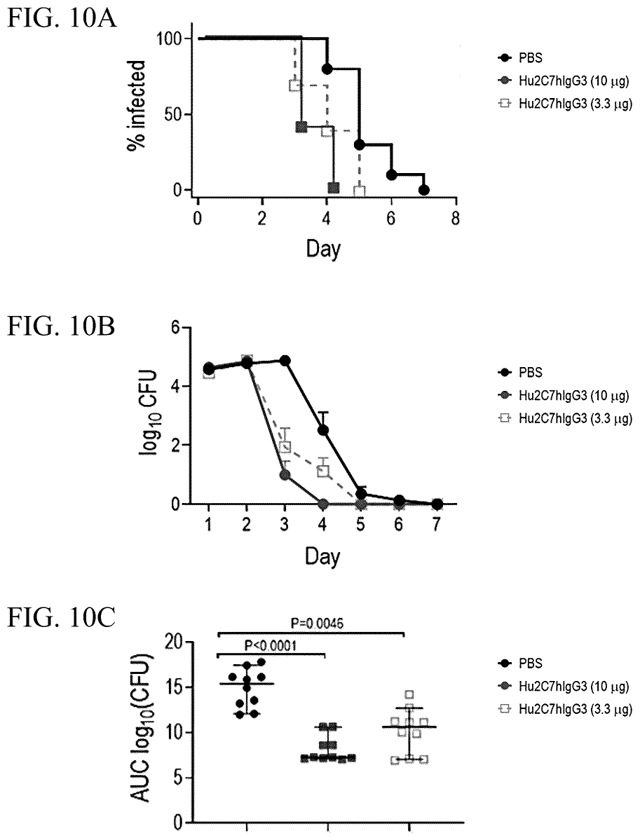

Efficacy of humanized mAb 2C7 (VH5 R→G with VL2 Y→G, A→G and W→G) hIgG3 Fc was evaluated in the mouse vaginal colonization model of gonorrhea. Dual transgenic (human factor H and C4BP) mice were inoculated with 2.2×10⁷ CFU of N. gonorrhoeae strain FA1090. Humanized 2C7 hIgG3 Fc was injected intravenously on day 1 after inoculation. Humanized 2C7 hIgG3 Fc showed efficacy in all three parameters (time to clearance, log₁₀ CFU vs time and AUC analysis) when administered at a dose of 10 or 3.3 μg/mouse, compared to PBS control (FIGS. 10A-10C).

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

The methods illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof. It is recognized that various modifications are possible within the scope of the invention claimed. Thus, it should be understood that although the present invention has been specifically disclosed by exemplary embodiments and optional features, modification and variation of the invention embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the methods. This includes the generic description of the methods with a proviso or negative limitation removing any subject matter from the genus, regardless of whether or not the excised material is specifically recited herein.

Other embodiments are within the following claims. In addition, where features or aspects of the methods are described in terms of Markush groups, those skilled in the art will recognize that the invention is also thereby described in terms of any individual member or subgroup of members of the Markush group.

1. A humanized antibody that binds the 2C7 epitope of Neisseria gonorrhoeae antigen, wherein the antibody comprises a variable heavy (VH) domain, a variable light (VL) domain, and a human Fc region,
wherein the VH domain comprises an amino acid sequence selected from the group consisting of SEQ ID NOs: 13-16, and
wherein the VL domain comprises an amino acid sequence of SEQ ID NO: 17.

2. The humanized antibody of claim 1, wherein the VH domain comprises the amino acid sequence of SEQ ID NO: 13 and the VL domain comprises the amino acid sequence of SEQ ID NO: 17.

3. The humanized antibody of claim 1, wherein the VH domain comprises the amino acid sequence of SEQ ID NO: 14 and the VL domain comprises the amino acid sequence of SEQ ID NO: 17.

4. The humanized antibody of claim 1, wherein the VH domain comprises the amino acid sequence of SEQ ID NO: 15 and the VL domain comprises the amino acid sequence of SEQ ID NO: 17.

5. The humanized antibody of claim 1, wherein the VH domain comprises the amino acid sequence of SEQ ID NO: 16 and the VL domain comprises the amino acid sequence of SEQ ID NO: 17.

6. The humanized antibody of claim 1, wherein the human Fc region is an IgG3 Fc region.

7. (cancelled)

8. The humanized antibody of claim 1, wherein the human Fc region is an IgG3 Fc region comprising one or more mutations that alters one or more Fc functions,
wherein the one or more Fc functions is selected from the group consisting of Fc clustering, C1q binding, complement-dependent cytotoxicity (CDC), antibody-dependent cell-mediated cytotoxicity (ADCC), and antibody dependent cellular phagocytosis (ADCP).

9. A pharmaceutical composition comprising the humanized antibody of claim 1 and a pharmaceutically acceptable carrier or diluent.

10. A nucleic acid encoding the humanized antibody amino acid sequence of claim 1.

11. A vector comprising the nucleic acid of claim 10.

12. A host cell comprising the vector of claim 11.

13. The host cell of claim 12, wherein the host cell is a prokaryotic cell or a eukaryotic cell,
wherein the prokaryotic cell is optionally E. coli, or
wherein the eukaryotic cell is selected from the group consisting of a yeast cell, a plant cell, an insect cell, and a mammalian cell, and wherein the plant cell is optionally a tobacco plant cell.

14. (canceled)

15. (canceled)

16. (canceled)

17. The host cell of claim 13, wherein the eurkaryotic cell is a human embryonic kidney (HEK) cell or a Chinese hamster ovary (CHO) cell.

18. The host cell of claim 17, wherein the host cell is a CHO cell.

19. A method of producing a humanized antibody that binds the 2C7 epitope of Neisseria gonorrhoeae antigen, the method comprising culturing a host cell in culture medium under conditions sufficient to produce the humanized antibody,
wherein the host cell comprises a vector encoding a humanized antibody that binds the 2C7 epitope of Neisseria gonorrhoeae antigen,
wherein the antibody comprises a variable heavy (VH) domain, a variable light (VL) domain, and a human Fc region,
wherein the VH domain comprises an amino acid sequence selected from the group consisting of SEQ ID NOs: 13-16, and
wherein the VL domain comprises an amino acid sequence of SEQ ID NO: 17.

20. A method of treating Neisseria gonorrhoeae infection in a subject in need thereof comprising administering to the subject a humanized antibody that binds the 2C7 epitope of Neisseria gonorrhoeae antigen,
wherein the antibody comprises a variable heavy (VH) domain, a variable light (VL) domain, and a human Fc region,
wherein the VH domain comprises an amino acid sequence selected from the group consisting of SEQ ID NOs: 13-16, and
wherein the VL domain comprises an amino acid sequence of SEQ ID NO: 17.

21. The method of claim 20, wherein the administered antibody improves clearance of the Neisseria gonorrhoeae infection.

22. The method of claim 20, wherein the administered antibody protects the subject from a subsequent Neisseria gonorrhoeae infection or improves clearance of the Neisseria gonorrhoeae infection and protects the subject from a subsequent Neisseria gonorrhoeae infection, optionally wherein the subject is a human.

23. (canceled)

24. The method of claim 20, wherein the Neisseria gonorrhoeae infection is resistant to antibiotics.

25. (canceled)

26. The method of claim 19, further comprising the step of purifying the antibody and formulating it as a pharmaceutical composition comprising a pharmaceutically acceptable carrier or diluent.
